(19) United States
(12) Patent Application Publication
(10) Pub. No.: US 2025/0208017 A1
(43) Pub. Date: Jun. 26, 2025

(54) METHOD OF DETERMINING THE SATURATION TIME OF AN ADSORBENT

(71) Applicant: CERAFILTEC GERMANY GmbH, Saarbruecken (DE)

(72) Inventors: Kay Gunther GABRIEL, Dubai (AE); Martin KASCHECK, St. Ingbert (DE)

(21) Appl. No.: 19/076,478

(22) Filed: Mar. 11, 2025

Related U.S. Application Data
(63) Continuation of application No. PCT/EP2023/072703, filed on Aug. 17, 2023.

(30) Foreign Application Priority Data
Sep. 15, 2022 (DE) 10 2022 123 613.7

(51) Int. Cl. G01N 15/08; G01L 11/00

(52) U.S. Cl. CPC G01N 15/0893; G01L 11/00

(57) ABSTRACT

A method determines the saturation duration of an adsorbent, including providing a filter coated with the adsorbent, where a medium to be filtered with a DOC content is present on the outside of the filter and the adsorbent forms a layer with an adsorbent mass on the outside of the filter and is suitable for adsorbing dissolved organic carbon from the medium to be filtered. A pump is provided and a pressure measuring device is arranged between the pump and the filter. The medium is pumped through the filter and the temporal course of filtration pressure is recorded. The filtration pressure increase rate is derived as a quotient of the filtration pressure increase per time unit. The saturation time is identified as the time of a significant change in the pressure increase rate, and an associated saturation duration is determined as the time from the start of pumping to saturation.

### CROSS REFERENCE TO RELATED APPLICATIONS

This application is a continuation of International Application No.: PCT/EP2023/072703, filed Aug. 17, 2023, which claims priority to German Application No. 10 2022 123 613.7, filed Sep. 15, 2022, the contents of each of which are incorporated by reference herein.

### FIELD OF THE INVENTION

The present invention relates to a method for determining the saturation duration of an adsorbent and a method for estimating the DOC content of a test medium to be filtered.

### BACKGROUND

The DOC content (DOC=Dissolved Organic Carbon) describes the proportion of dissolved organic carbon in a liquid medium. As a so-called organic sum parameter, the DOC content is therefore a measure of the total of all dissolved organic compounds and represents an important parameter for characterising water quality.

Together with the particulate (undissolved) organic carbon (POC=Particular Organic Carbon) and the volatile organic carbon (VOC=volatile organic carbon), the DOC component forms the total organic carbon (TOC=total organic carbon). Particulate organic carbon is usually separated using a coarse microfilter with a pore diameter of around 0.45 μm.

The direct measurement of organic sum parameters poses a challenge in practice. For example, for the sum parameter biological oxygen demand (BOD), a water sample must be measured over a test procedure lasting several days. In the case of chemical oxygen demand (COD), several hours are usually required before the measurement result is available. It is therefore not possible to react directly to short-term changes in the concentration of organic substances in the water for operational purposes.

The situation is similar with the measurement of the TOC content and the DOC content. The various measurement methods are described, for example, in the standardisation DIN EN1484, ASTM D4839, ASTM D4779.

According to a first measurement method known in the prior art, the TOC or DOC content is determined by heating a sample of the liquid to be determined to approx. 700 to 1,000° C., usually with the addition of oxygen, using a combustion process, so that the organic carbon it contains is completely converted to carbon dioxide (CO2). The mass of the CO2 is then determined using so-called non-dispersive infrared detectors (NDIR) and the TOC or DOC is then calculated.

In the so-called UV persulphate method, on the other hand, acid is first added to a heated sample to convert the inorganic carbon into CO2. CO2 is then expelled from the sample using nitrogen. Persulphate is then added to the sample and the sample is irradiated with UV light. The organic carbon contained is then converted into CO2 in a heated reactor and expelled from the sample using nitrogen. The mass of the CO2 is then also determined using an NDIR so that the TOC or DOC can be calculated.

Both measurement methods provide very precise quantitative results. However, this high level of accuracy comes at the cost of several complex (laboratory) work steps, which is time-consuming and expensive.

Alternatively, the DOC content or the TOC content can be determined using the so-called UV adsorbance signal measurement (turbidity-compensated signal), also known as SAK (spectral adsorption coefficient). This method utilises the fact that there is a correlation between the SAK value and the DOC content.

The measurement method is based on the fact that numerous dissolved organic carbons have aromatic rings or double bonds that adsorb UV light at a wavelength of 254 nm. By determining the spectral adsorption coefficient (SAK) of the sample in this wavelength range, the DOC content can be approximated quickly and cost-effectively using a simple photometric or calorimetric measuring principle. The disadvantage, however, is that the correlation between the SAK value and the DOC content is water-specific, which means that the correlation must be determined individually for each measuring site in advance using comparative tests (so-called jar-tests) and checked regularly. The correlation can also changes seasonally, which requires a complex conversion from SAK value to TOC.

For water (or wastewater) to be filtered, it is known that an increasing DOC content has a negative effect on the performance of the filter. The main reason for this is the build-up of dissolved organic carbon on the surface of the filter, known as organic fouling:

The dissolved organic carbons can pass through the (micro or ultrafiltration) filter on the one hand, but can also accumulate on the surface of the respective filter on the other. Many dissolved organic carbons, especially those of natural origin such as humic substances, proteins or polysaccharides, have hydrophobic (water-repellent) characteristics. If these accumulate on the surface of the filter, the filter also becomes more hydrophobic (hydrophobisation of the filter). As a result, the water experiences more resistance when passing through the filter pores of the filter. With a constant flow rate (volume flow) through the filter, this leads to an increase in filtration pressure, which is accompanied by a reduction in filter performance. The accumulation of dissolved organic carbon on the surface of the filter typically occurs continuously during filtration, which means that the filtration pressure also increases continuously over the filtration time (see FIG. 2, straight line G1 with straight line gradient m1) and thus the filter performance decreases continuously.

In order to minimise this negative influence on filter performance, DOC-reducing substances (adsorbents) are usually used upstream of the filter in water and wastewater treatment. For this purpose, metal-based coagulants, such as precipitating iron chlorides, aluminium sulphates, but also powdered activated carbon (PAK), are continuously dosed into the medium to be filtered upstream of the filter in order to reduce the DOC content (DOC concentration). If dissolved organic carbon has accumulated on these adsorbents, it can no longer accumulate on the filter and thus reduce the filter performance. In this way, the increase in filtration pressure over the filtration time can be reduced (see FIG. 2, straight line G2 with straight line gradient m2 compared to straight line G1 with straight line gradient m1).

In this context, the publication DE 2812 819 A1 describes an exemplary process for drinking water treatment in which phosphate dissolved in the water to be treated is precipitated and separated by adding the flocculant iron (III) chloride.

The DOC-reducing substances (adsorbents) are typically dosed in terms of quantity and time of addition in such a way that DOC absorption is completed before the DOC-reducing substances come into contact with the filter. This is usually achieved by dosing the DOC-reducing substances into the medium to be filtered (water) in a high-speed mixer and then keeping them in suspension in a retention tank until DOC absorption is complete and only then does the water come into contact with the filter. At the time of contact with the filter, the DOC-reducing substances are ideally used up, i.e. already saturated with DOC, so that no further DOC uptake takes place. The maximum absorption quantity or adsorption capacity is therefore achieved before reaching the filter, so that the remaining “free” DOC content in the water is reduced. As a result, the filter is better protected against organic fouling.

During the filtration time, a filter cake of saturated DOC-reducing substances (adsorbent) continuously grows on the filter surface of the filter. As the DOC-reducing substances are already saturated, no further DOC uptake by the saturated adsorbent takes place. This means that the “free” DOC content in the water, in the filter itself and in the filtered water is constant. The quantitative dosage and the saturation duration of the adsorbent are usually determined and specified beforehand by means of comparative tests (jar-tests).

In this context, publication CH707684A2 discloses a process for water purification in which the filter cake (“retained adsorbent”) deposited on the filter surface is removed by backwashing and at least partially reused.

The publication DE 10 2014 107 489 A1 describes an adsorptive filter process for water treatment in which an “adsorption material in the form of a spherical activated carbon” with special properties in terms of total pore volume and hydrophilicity is used.

Against this background, the present invention is based on the task of providing a method for determining the saturation duration of an adsorbent and, in particular, a method for estimating the DOC content of a medium to be filtered with improved practical suitability, in particular with regard to ease of use during operation, exact dosing of the adsorbent and process efficiency.

### SUMMARY

This task may be solved by the method for determining the saturation duration of an adsorbent according to this disclosure.

The method for determining the saturation duration of the adsorbent comprises the following steps:
A) providing a filter coated with the adsorbent, wherein
   the filter has a filter outer side with a filter outer surface and a filter inner side, a medium to be filtered with a DOC content is present on the filter outer side and the filtrate can be discharged via the filter inner side,
   the adsorbent forms a layer with an adsorbent mass on the outside of the filter and is suitable for adsorbing dissolved organic carbon from the medium to be filtered (in particular water),
B) providing a pump and a pressure measuring device arranged between the pump and the filter, wherein
   the pump is suitable for pumping the medium to be filtered through the filter at a pump flow rate, and
   the pressure measuring device is suitable for determining the filtration pressure (measured against the ambient pressure),
C) starting of pumping the medium to be filtered through the filter,
D) recording the temporal course of the filtration pressure (during the pumping of the medium to be filtered through the filter) and deriving the filtration pressure increase rate as a quotient of the filtration pressure increase per time unit, and
E) identifying the time of saturation of the adsorbent (saturation time) as the time of a significant change in the filtration pressure increase rate and determine an associated saturation duration as the time from the start of pumping to the saturation time.

The filter is therefore coated with an initially (at least largely) unsaturated adsorbent, which adsorbs undissolved organic hydrocarbons when the medium to be filtered flows through it. After the pumping is started, the undissolved organic hydrocarbon (DOC) constantly accumulates on the adsorbent until the adsorbent reaches saturation point. Ideally, the (initially unsaturated) adsorbent on the filter can completely absorb the entire DOC content of the medium to be filtered by the time it reaches saturation, so that the filter is also completely protected against organic fouling. In this ideal case, the filtration pressure remains constant over the filtration time, the filtration pressure increase rate is zero and there is no drop in filter performance (see FIG. 2, straight line G3 with straight gradient m3). In practice, however, this ideal case can only rarely be achieved. Instead, the initially unsaturated adsorbent on the filter only absorbs a very large proportion of the DOC load of the medium (to be filtered) up to the point of saturation, so that the medium passing the filter surface only has a low DOC content and the filter is therefore only marginally exposed to organic fouling. Accordingly, the filtration pressure only increases very slightly over the filtration time (see FIG. 2, straight line G4), the filtration pressure increase rate is very low (see FIG. 2, straight line gradient m4) and the drop in filter performance is correspondingly small.

The time at which the filtration pressure increase rate changes (i.e. the inflection point of the temporal filtration pressure curve) corresponds to the saturation time of the adsorbent. The saturation point is therefore the point in time during filtration at which the maximum adsorption capacity of the adsorbent is reached. This means that after reaching the saturation point, no (or only insignificantly little) further DOC load of the medium can be absorbed by the adsorbent and therefore the organic carbon dissolved in the medium passes through the adsorbent layer and consequently hits the filter and hydrophobises it more strongly or more quickly. This leads to a greater increase in filtration pressure over the filtration time and is reflected in a significant change in the filtration pressure increase rate, as the filtration pressure increase rate after reaching the saturation point is significantly greater than before reaching the saturation point.

A significant change (increase) in the filtration pressure increase rate within the meaning of the present invention is typically present if the filtration pressure increase rate of the current time interval is at least 10% greater than the filtration pressure increase rate of the previous time interval, whereby it may be expedient to consider changes as significant only if the filtration pressure increase rate of the current time interval is at least 20%, 50% or 100% greater than that of the previous time interval.

It has been found that this significant change can be determined particularly reliably if the current time interval has a length of at least one minute and the previous time interval corresponds to the duration from the start of pumping to the start of the current time interval and thus the associated filtration pressure increase rate describes the average previous filtration pressure increase rate.

The time of saturation or the duration of saturation of the adsorbent can therefore be determined directly during ongoing filter operation. If the method is used to recognise that the adsorbent is saturated, this information can be used directly to control the further filtration process, for example by interrupting the filtration and renewing the adsorbent. In this way, the adsorption capacity of the adsorbent can be optimally utilised and waste minimised.

Common pressure measuring devices, such as pressure sensors or pressure gauges, can be used to measure the filtration pressure. The pressure measuring device is installed between the filter and the pump and measures the filtration pressure against the ambient pressure. In so-called pressure-operated filters, the pump and the filter are arranged upstream of the filter (i.e. on the pressure side of the filter) (see FIG. 1a), whereas in suction-operated filters the pump and the filter are arranged downstream of the filter (i.e. on the filtrate side of the filter) (see FIG. 1b). During filtration, water is transported through the filter and the filtration pressure upstream of the filter is continuously measured.

The method makes it possible to determine the saturation duration of the adsorbent via the course of the filtration pressure. Since the filtration pressure is a parameter that is easy to determine and is often already recorded for other reasons, the saturation duration can be determined in this way with minimum effort.

Furthermore, the method enables a more efficient filtering process, since it is not necessary to allow the medium to be filtered to remain in a retention basin until the dissolved organic hydrocarbons are bound by the adsorbent, as is common in the prior art.

Based on the method just described for determining the saturation duration of the adsorbent, the method for estimating the actual DOC content (i.e. for determining the approximate actual DOC content) of a test medium to be filtered comprises the following steps:
F) determining the actual saturation duration of the test medium to be filtered for determining the saturation duration of the adsorbent, and
G) estimating the actual DOC content (i.e. determining the approximate actual DOC content) of the test medium to be filtered by dividing a calibration constant by the actual saturation duration.

After the saturation duration has been determined as the actual saturation duration for the test medium to be filtered with an unknown DOC content (actual DOC content), the actual DOC content can be estimated in this way (i.e. approximately determined) by dividing the calibration constant by the determined actual saturation duration.

The invention is based on the realisation that the DOC content can be directly and approximately determined or estimated via the correlating measurement parameter of the saturation duration, whereby-all other things being equal-a shorter saturation duration indicates a higher DOC content

In this way, the DOC content of the test medium to be filtered can be estimated and monitored directly during ongoing filter operation with minimal effort and provide a valuable basis of information for further process control. The method can therefore also be used advantageously in the so-called main flow of a filtration process, as the required filtration pressure measurement is usually already installed in the main flow of the filtration process anyway.

According to a preferred embodiment of the method, the calibration constant is determined in the process,
by determining the saturation duration (calibration saturation duration) for a calibration medium with a known DOC content (calibration DOC content) according to the method described above for determining the saturation duration, and
the calibration constant is a function of the calibration DOC content and the calibration saturation duration.

To determine the calibration constant, the (calibration) DOC content and the (calibration) saturation duration must be determined for a calibration medium that is as similar as possible to the medium to be filtered. It is therefore advisable to use the medium from the same source as the test medium to be filtered as the calibration medium. Using the standard measurement methods described above (e.g. according to standardisation DIN EN1484, ASTM D4839, ASTM D4779), the DOC content of the calibration medium is then determined and used as the calibration DOC content and then the saturation duration for the calibration medium is determined using the method described above for determining the saturation duration.

The quality of the medium to be filtered (especially water) and therefore also the DOC content can change permanently over time. Therefore, the calibration DOC content must be checked at regular intervals and the associated calibration saturation duration must be redetermined. For this purpose, a sample can be taken at regular intervals from the source that supplies the test medium to be filtered, which is then used as the calibration medium.

Advantageously, the actual saturation duration of the test medium to be filtered is determined and the calibration saturation duration of the calibration medium is determined at identical pump volume flows, identical filter outer surfaces and identical adsorbent quantities using the same adsorbent.

This is because the inventors have recognised that under these conditions the DOC content and the saturation duration are approximately indirectly proportional to each other, so that-according to a further preferred embodiment of the method according to the invention-the calibration constant is the product of the calibration DOC content and the calibration saturation duration.

The pump volume flows, the adsorbent type, the adsorbent quantity and the filter outer surface are therefore defined process variables. The determined calibration saturation duration is only valid for these defined process variables.

According to a further preferred embodiment of the invention, the filter coated with an adsorbent is provided according to step A) by applying the unsaturated adsorbent to the outside of the filter by means of a precoat filtration process.

For this purpose, the unsaturated adsorbent, which is to be deposited on the filter as a coating, is first completely added to a precoat medium and mixed with it. The precoat medium is then pumped through the filter so that the unsaturated adsorbent is deposited evenly and homogeneously on the outside of the filter and forms an (adsorbent) layer on the outside of the filter. Preferably, the thickness of the adsorbent layer is between 0.1 and 3 mm.

The medium to be filtered can be used as the precoat medium. The adsorbent then adsorbs DOC from the medium to be filtered while it is being precoated onto the filter surface and is therefore no longer completely unsaturated in the strict sense when the adsorbent layer has formed on the filter. However, as this precoating process takes place relatively quickly, it can be assumed that the adsorbent is at least largely unsaturated by the time the precoating process is complete and the adsorbent layer has formed

Once filtration is complete, the filter is backwashed according to a further preferred embodiment by separating the (then saturated) adsorbent from the outside of the filter by backwashing. In this way, the solids deposited on the filter, including the adsorbent, can be completely removed and removed from a filtration tank (in which the filter is located). After removal, the filtration tank is refilled with the precoat medium and the adsorbent and the precoat process begins again.

According to a further preferred embodiment of the process according to the invention, the filter is designed as a micro or ultrafiltration filter and has filter pores with a filter pore diameter of 0.05 to 2.0 μm, and the adsorbent has particles with a particle size of 5 to 500 μm. The particle size of the adsorbent is therefore significantly larger than the pore diameter of the filter. This means that no adsorbent can be deposited in the pores and block the pores or even penetrate through the filter. Instead, a loose adsorbent layer forms on the surface of the filter in an advantageous manner.

Advantageously, the filter is designed as a ceramic filter, glass filter, metal filter or plastic filter. Filters with pore diameters in the desired micro or ultrafiltration filter range can be manufactured from all of these materials.

The adsorbent advantageously comprises powdered activated carbon (PAK) or a metal-based coagulant, in particular iron chlorides or aluminium sulphates.

### BRIEF DESCRIPTION OF THE DRAWING

The methods according to the invention are explained in more detail below with reference to the drawing.

FIGS. 1a and 1b each schematically show a filtration device comprising a pump, a pressure measuring device and a filter configuration, with FIG. 1a showing a pressure-operated filter and FIG. 1b a suction-operated filter,

FIG. 2 shows schematic filtration pressure curves for filtration devices according to FIG. 1a or 1b, and

FIG. 3 shows measured filtration pressure curves for a filtration device according to FIG. 1b.

### DETAILED DESCRIPTION OF THE PREFERRED EMBODIMENTS

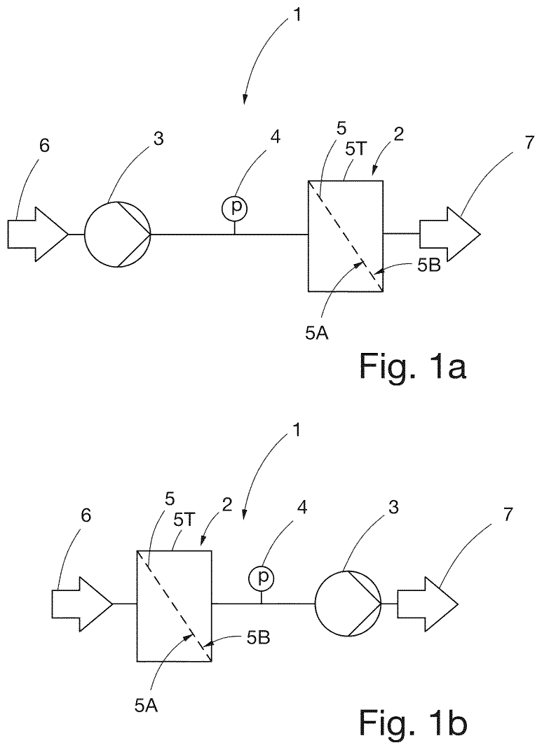

FIGS. 1a and 1b each schematically show a filtration device 1 comprising a filter configuration 2, a pump 3 and a pressure measuring device 4 arranged between the filter configuration 2 and the pump 3. The filter configuration 2 comprises a filter 5 with a filter outer side 5A and a filter inner side 5B and a filtration tank 5T in which the filter 5 is arranged.

FIG. 1a shows a pressure-operated filter. The pump 3 and the pressure measuring device 4 are arranged upstream of the filter configuration 2, i.e. on the side of the filter 5 facing the outside of the filter 5A.

FIG. 1b, on the other hand, shows a suction-operated filter. Here, the pump 3 and the pressure measuring device 4 are arranged downstream of the filter configuration 2, i.e. on the side of the filter 5 arranged on the inside of the filter 5B.

The pump 3 is suitable for pumping (or sucking) a medium to be filtered (in particular water) from the source 6 through the filter (i.e. from the outside of the filter 5A to the inside of the filter 5B) to the sink 7.

In the pressure-operated filter according to FIG. 1a, the pressure measuring device 4 determines the filtration pressure p (compared to the ambient pressure) that is set in the medium to be filtered upstream of the filter configuration 2. In the suction-operated filter as shown in FIG. 1b, the pressure measuring device 4 determines the filtration pressure p (compared to the ambient pressure), which is set in the filtrate downstream of the filter configuration 2.

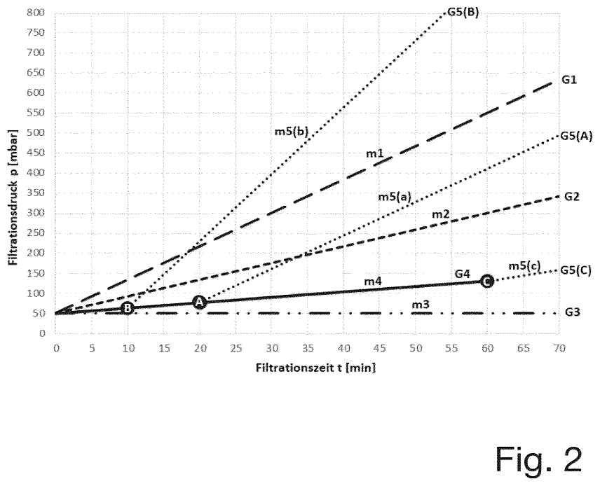

FIG. 2 shows several schematic curves of the filtration pressure p in mbar over the filtration time t in minutes (min) for filtration devices 1 according to FIG. 1a or 1b.

The straight line G1 represents the schematic curve of the filtration pressure p over the filtration time t when a medium to be filtered with a certain DOC content DOC_1 is pumped through the filter 5 of the filter device 2 and no measures are taken to adsorb the DOC. Dissolved organic carbon is deposited on the outside 5A of the filter 5 and leads to organic fouling. The medium to be filtered (water) flowing through the filter 5 is met with ever greater resistance due to the progressive hydrophobisation of the filter 5, which is reflected in a continuously increasing filtration pressure p. The filtration pressure increase rate represents the quotient of the filtration pressure increase per time unit and corresponds graphically to the straight line gradient of the respective straight line.

The filtration pressure curve shown schematically by the straight line G2 results when an adsorbent is first added to the medium to be filtered and the medium only reaches the filter 5 after the DOC content of the medium has been reduced by the adsorbent. Here too, dissolved organic carbon is deposited on the filter surface (or on the outside of the filter 5A) with increasing filtration time t, leading to organic fouling and a continuously increasing filtration pressure p. However, the filtration pressure increase rate m2 is lower with G2 than with G1 because the DOC content of the medium hitting the filter is lower. The filter performance loss is therefore lower.

The straight lines G3, G4 and G5(A), G5(B) and G5(C), on the other hand, schematically illustrate filtration pressure curves that occur when implementations of the methods are used. At the beginning of the filtration time t (i.e. at t=0), the pump is started in order to pump the medium to be filtered through the filter and the filter 5 of the filtration device 2 is coated with an unsaturated adsorbent. The unsaturated adsorbent forms an (adsorbent) layer with an adsorbent mass on the outside of the filter 5A and is suitable for adsorbing dissolved organic carbon from the medium to be filtered.

The ideal curve according to straight line G3 results in the case where the adsorbent layer completely filters the DOC content out of the medium to be filtered. The medium hitting the outside 5A of the filter 5 is therefore completely free of DOC, so that organic fouling is completely prevented. The filtration pressure p remains constant, the filtration pressure increase rate m3 is therefore zero and filtration performance losses can be completely prevented (up to the saturation point) (the saturation point of the adsorbent is not reached within the filtration time of 70 min shown in FIG. 2). However, this ideal case will only be encountered very rarely in practice.

In practice, the case outlined by the straight lines G4 and G5(A), G5(B) and G5(C) is more likely to occur. In this case, the adsorbent layer adsorbs a large proportion of the DOC content of the medium to be filtered-but not all of it. The non-adsorbed part of the DOC component thus reaches the outside of the filter 5A, is deposited there (at least partially) and causes organic fouling, which is reflected in a hydrophobisation of the filter and thus in a continuous increase in the filtration pressure p with the filtration pressure increase rate m4. After a certain filtration time t, the adsorption capacity of the adsorbent layer is exhausted and no more DOC can be removed from the medium to be filtered. This point in time (illustrated in FIG. 2 as A, B, C) is also referred to as the saturation point. Consequently, after the saturation point, more DOC arrives at the outside of the filter per unit of time and is deposited there, which accelerates the increase in filtration pressure p. As a result, the filtration pressure increase rate m5(a), m5(b), m5(c) is greater after the respective saturation point than before the saturation point, so that the saturation point appears as a kink in the filtration pressure curve.

For a medium to be filtered with a first DOC proportion DOC_A, this results in a saturation time A at the filtration time t=20 min, the saturation duration of the adsorbent is therefore 20 min. For a DOC proportion DOC_B that is twice as large as the DOC proportion DOC_A, the saturation duration is halved to 10 min-under otherwise identical conditions (see saturation time B). For a DOC proportion DOC_C that is only one third of the DOC proportion DOC_A, the saturation duration triples to 60 min (see saturation time C).

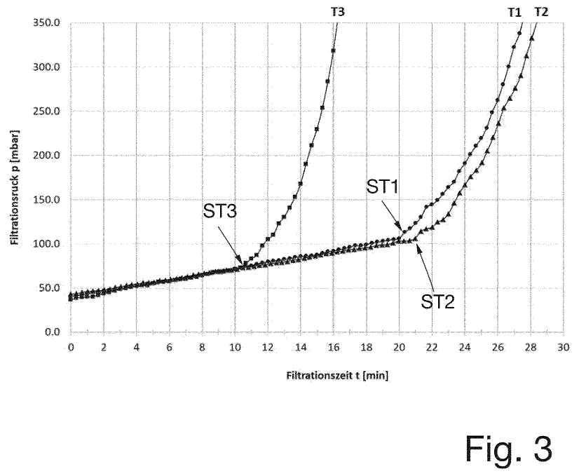

In contrast to FIG. 2, FIG. 3 does not show schematic filtration pressure curves, but three real filtration pressure curves T1 to T3 (measurements) which were obtained using the methods in a filtration device as shown in FIG. 1b. All the filtration pressure curves T1 to T3 shown have in common that the pump volume flow was 250 litres per hour, that the external filter surface area was 1 m² and that the adsorbent used was powdered activated carbon, which initially (i.e. at filtration time t=0) formed an adsorbent layer of 10 g/m² of external filter surface area.

For the filtration pressure curves T1 and T2, river water with a DOC content of 8.1 mg/l was used as the medium to be filtered; for the filtration pressure curve T3, river water with a DOC content of 15.2 mg/l was used. The measured filtration pressure p was determined approximately three times per minute after starting the pump and plotted against the elapsed filtration time. It can be seen that the filtration pressure curves T1 to T3 initially increase at an almost constant filtration pressure increase rate until the respective saturation points ST1, ST2 and ST3 are reached and the filtration pressure increase rates change or increase significantly. The saturation times ST1 and ST2 are at a filtration time of approx. 20.5 min and 21 min respectively. The difference between the filtration pressure curves T1 and T2 is largely due to measurement inaccuracies. The saturation point ST3 is located at a filtration time of around 10.5 min. This means that the associated saturation duration of the filtration pressure curve T3 is around half as long as for the filtration pressure curves T1 and T2, while the DOC content of the filtration pressure curve T3 is twice as long as for T1 and T2. The measured values thus confirm the finding that the saturation duration-under otherwise constant conditions-is indirectly proportional to the DOC content.

In all three measurements T1 to T3, the DOC content in the filtrate (i.e. the filtered water) was in the range of 0.94-1.11 mg/l before reaching the saturation point. This results in an adsorption rate of 7.16 and 7.07 mg/l for measurements Tl and T2 respectively and an adsorption rate of 14.09 mg/l for measurement T3. In measurement T3, the adsorbent also absorbs twice as much DOC due to the almost doubled DOC content of the medium to be filtered (river water). As a result, the filtration time to saturation is halved from 20.5 or 21.0 min for measurement T1 or T2 to just 10.5 min for measurement T3.

After reaching the saturation point of the adsorbent, the DOC content in the filtrate (filtered water) approached the DOC content of the medium to be filtered (river water). The DOC content in the filtrate (filtered water) was in the range of 7.61-7.33 mg/l for measurements T1 and T2, for measurement T3 the corresponding value was 14.14 mg/l.

After the saturation point, the filter itself comes into direct contact with river water with a higher DOC content. This allows more and faster DOC to accumulate as (organic) fouling on the surface of the filter, which results in a faster increase in filtration pressure. It can also be clearly seen that due to the DOC content in the filtrate (filtered water) of measurement T3 being approximately twice as high, the associated filtration pressure increase rate after the saturation point is also approximately twice as high as in measurements T1 and T2.

What is claimed is:

1. A method for determining the saturation duration of an adsorbent comprising the following steps:
A) providing a filter (5) coated with the adsorbent, wherein
the filter (5) has a filter outer side (5A) with a filter outer surface and a filter inner side (5B), a medium to be filtered with a DOC content is present on the filter outer side (5A) and the filtrate can be discharged via the filter inner side (5B),
the adsorbent forms a layer with an adsorbent mass on the outside of the filter (5A) and is suitable for adsorbing dissolved organic carbon from the medium to be filtered,
B) providing a pump (3) and a pressure measuring device (4) arranged between the pump (3) and the filter (5), wherein
the pump (3) is suitable for pumping the medium to be filtered through the filter (5) at a pump flow rate, and
the pressure measuring device (4) is suitable for determining the filtration pressure (p),
C) starting of pumping the medium to be filtered through the filter (5),
D) recording the temporal course of the filtration pressure (p) and deriving the filtration pressure increase rate as a quotient of filtration pressure increase per time unit, and
E) identifying the saturation time (A, B, C, ST1, ST2, ST3) of the adsorbent as the time of a significant change in the filtration pressure increase rate and determine an associated saturation duration as the time from the start of pumping to the saturation time.

2. A method for estimating the actual DOC content of a test medium using to be filtered using the method of claim 1, comprising the following steps:
F) determining the actual saturation time of the test medium to be filtered according to steps A-E, and
G) determining the approximate actual DOC content of the test medium to be filtered by dividing a calibration constant by the actual saturation duration.

3. The method of claim 2, wherein the calibration constant is determined:
by determining the saturation duration (calibration saturation duration) for a calibration medium with a known DOC content (calibration DOC content) according steps A-E, and
the calibration constant is a function of the calibration DOC content and the calibration saturation duration.

4. The method of claim 3, wherein the determination of the actual saturation duration of the test medium to be filtered and the determination of the calibration saturation duration of the calibration medium are carried out at identical pump volume flows, identical filter outer surfaces and identical adsorbent quantities using the same adsorbent.

5. The method of claim 4, wherein the calibration constant is the product of the calibration DOC content and the calibration saturation duration.

6. The method of claim 1, wherein the filter (5) coated with an adsorbent is provided according to step A) by applying the unsaturated adsorbent to the outside of the filter (5A) by means of a precoat filtration process.

7. The method of claim 1 with the additional step:
H) separating the adsorbent from the outside of the filter (5A) by backwashing.

8. The method of claim 1, wherein:
the filter (5) has filter pores with a filter pore diameter of 0.05 to 2.0 μm, and
the adsorbent has particles with a particle size of 5 to 500 μm.

9. The method of claim 1, wherein the filter (5) is designed as a ceramic filter, glass filter, metal filter or plastic filter.

10. The method of claim 1, wherein the adsorbent comprises powdered activated carbon or a metal-based coagulant, in particular ferric chlorides or aluminium sulphates.
